(19) United States
(12) Patent Application Publication
(10) Pub. No.: US 2024/0415412 A1
(43) Pub. Date: Dec. 19, 2024

(54) METHOD AND APPARATUS FOR CONTROLLING AND CONFIGURING GUIDE ROBOT SYSTEM FOR CORRECTION AND EVALUATION OF WALKING POSTURE

(71) Applicant: ELECTRONICS AND TELECOMMUNICATIONS RESEARCH INSTITUTE, Daejeon (KR)

(72) Inventors: Young Jin KWON, Daejeon (KR); Jae Chul KIM, Daejeon (KR)

(21) Appl. No.: 18/653,834

(22) Filed: May 2, 2024

(30) Foreign Application Priority Data
Jun. 16, 2023 (KR) 10-2023-0077727
Aug. 11, 2023 (KR) 10-2023-0105698

(51) Int. Cl. A61B 5/11; A61B 5/00; B25J 19/02

(52) U.S. Cl. CPC A61B 5/112; A61B 5/1128; A61B 5/706; B25J 19/021

(57) ABSTRACT

The present invention relates to a method and apparatus for controlling and configuring a guide robot system for correction and evaluation of walking posture. A method of analyzing and evaluating gait according to an embodiment of the present disclosure may comprise: projecting a guide footprint at a pre-configured point based on a user's walking direction; measuring a user's footprint in walking performed by a user based on a position of the guide footprint; and performing analysis and evaluation of the user's footprint based on the guide footprint.

### CROSS-REFERENCE TO RELATED APPLICATIONS

This application claims the benefit of earlier filing date and right of priority to Korean Application No. 10-2023-0077727, filed on Jun. 16, 2023 and Korean Application No. 10-2023-0105698, filed on Aug. 11, 2023, the contents of which are all hereby incorporated by reference herein in their entirety.

### TECHNICAL FIELD

The present disclosure relates to a method and apparatus for controlling and configuring a guide robot system for correction and evaluation of walking posture, particularly gait.

### BACKGROUND

The method of walking is naturally acquired at birth, and the walking posture, or gait, may differ between people depending on physical ability and will. Additionally, changes in walking speed and toe-dragging gait can be used to diagnose diseases.

In this regard, in order to measure and evaluate gait, Technology was mainly used to evaluate and predict walking ability by performing a walking motion on a pressure sensor or analyzing movement using sensors such as pressure or Inertial Measurement Unit (IMU) mounted on shoes. However, in the case of such technology, there is a problem in that it is difficult to provide the results/feedback of evaluating walking ability to the subject in real time in order to correct walking.

### SUMMARY

The technical object of the present disclosure is to provide a method and apparatus for controlling and configuring a guide robot system for correction and evaluation of walking posture, particularly gait.

Specifically, the technical object of the present disclosure is to provide a method and apparatus that may profile a user's gait and immediately train an improved gait to improve or maintain walking ability.

The technical objects to be achieved by the present disclosure are not limited to the above-described technical objects, and other technical objects which are not described herein will be clearly understood by those skilled in the pertinent art from the following description.

A method of analyzing and evaluating gait according to an aspect of the present disclosure may comprise: projecting a guide footprint at a pre-configured point based on a user's walking direction; measuring a user's footprint in walking performed by a user based on a position of the guide footprint; and performing analysis and evaluation of the user's footprint based on the guide footprint. Herein, the guide footprint may be determined based on gait data pre-stored for the user, and the analysis and evaluation of the user's footprint may be based on a scoring method performed on a step-by-step basis according to the overlapping area of the user's footprint based on the guide footprint and a direction of the user's footprint.

An apparatus for analyzing and evaluating gait according to an additional aspect of the present disclosure may comprise a processor and a memory, and the processor may be configured to: project a guide footprint at a pre-configured point based on a user's walking direction; measure a user's footprint in walking performed by a user based on a position of the guide footprint; and perform analysis and evaluation of the user's footprint based on the guide footprint. Herein, the guide footprint may be determined based on gait data pre-stored for the user, and the analysis and evaluation of the user's footprint may be based on a scoring method performed on a step-by-step basis according to the overlapping area of the user's footprint based on the guide footprint and a direction of the user's footprint.

As one or more non-transitory computer readable medium storing one or more instructions according to an additional aspect of the present disclosure, the one or more instructions are executed by one or more processors and control an apparatus for analyzing and evaluating gait to: project a guide footprint at a pre-configured point based on a user's walking direction; measure a user's footprint in walking performed by a user based on a position of the guide footprint; and perform analysis and evaluation of the user's footprint based on the guide footprint. Herein, the guide footprint may be determined based on gait data pre-stored for the user, and the analysis and evaluation of the user's footprint may be based on a scoring method performed on a step-by-step basis according to the overlapping area of the user's footprint based on the guide footprint and a direction of the user's footprint.

In various aspects of the present disclosure, the method may further comprise: moving to other point in front of the user using a pre-configured movement control method according to walking direction of the user; and performing analysis and evaluation of a user's subsequent footprint by projecting a subsequent guide footprint and measuring the user's subsequent footprint at the other point.

Additionally, in various aspects of the present disclosure, the pre-stored gait data may correspond to data profiled based on one or more of data on a body of the user or data on gait correction previously performed by the user. In this regard, the data on a body of the user may be input through an interface of a display mounted on a device that projects the guide footprint or an interface of a terminal linked to the device.

Additionally, in various aspects of the present disclosure, a function of projecting the guide footprint may be designed to enable correction based on pre-configured reference footprints and image recognition by one or more cameras.

Additionally, in various aspects of the present disclosure, a function of measuring the guide footprint may be designed to enable correction based on a pre-configured reference pattern and image recognition by one or more cameras.

Additionally, in various aspects of the present disclosure, the analysis of the user's footprint may be performed by additionally applying one or more of an entry angle, time of occupying a ground, or drag information of foot of the user.

Additionally, in various aspects of the present disclosure, a pre-configured special effect may be additionally provided for the guide footprint depending on the degree of coincidence between the guide footprint and the user's footprint. In this regard, the pre-configured special effect may be configured in association with gait data pre-stored for the user or content of an indoor interlocking system.

According to the present disclosure, a method and appratus for controlling and configuring a guide robot system for correction and evaluation of walking posture, particularly gait, may be provided.

According to the present disclosure, a method and apparatus may be provided that can profile a user's gait and immediately train an improved gait to improve or maintain walking ability.

According to the present disclosure, it has the effect of achieving the purpose of maintaining body balance by maintaining correct walking posture and supporting the recognition of diseases or physical ability decline by analyzing accumulated walking postures and providing evaluation results of suspected diseases or physical abilities.

According to the present disclosure, it is possible to correct and evaluate the gait of each user while wearing the shoes they are wearing, by providing a structure that does not use the shape of a shoe or a pressure plate.

Effects achievable by the present disclosure are not limited to the above-described effects, and other effects which are not described herein may be clearly understood by those skilled in the pertinent art from the following description.

### BRIEF DESCRIPTION OF THE DRAWINGS

FIG. 1 illustrates an example of a guide robot system for gait posture correction and evaluation according to an embodiment of the present disclosure.

FIG. 2 illustrates another example of a guide robot system for gait posture correction and evaluation according to an embodiment of the present disclosure.

FIG. 3 illustrates a control method of a guide robot system according to an embodiment of the present disclosure.

FIG. 4 illustrates a block diagram of FIG. 4 illustrates a block diagram of a footstep guide-evaluation system by a guide robot according to an embodiment of the present disclosure.

FIG. 5 illustrates a correction function for providing guide footprints and measuring user footsteps according to an embodiment of the present disclosure.

FIG. 6 illustrates a method of measuring guide footprints and user footprints for gait posture correction and evaluation according to an embodiment of the present disclosure.

FIG. 7 illustrates an overlap state between guide footprints and user footprints according to an embodiment of the present disclosure.

FIG. 8 illustrates footstep analysis according to an embodiment of the present disclosure.

FIG. 9 illustrates guide footprint projection and movement of the gait guide robot according to an embodiment of the present disclosure.

FIG. 10 illustrates an operational flowchart of a method for analyzing and evaluating a user's gait according to an embodiment of the present disclosure.

FIG. 11 is a block diagram illustrating a device according to an embodiment of the present disclosure.

### DETAILED DESCRIPTION

As the present disclosure may make various changes and have multiple embodiments, specific embodiments are illustrated in a drawing and are described in detail in a detailed description. But, it is not to limit the present disclosure to a specific embodiment, and should be understood as including all changes, equivalents and substitutes included in an idea and a technical scope of the present disclosure. A similar reference numeral in a drawing refers to a like or similar function across multiple aspects. A shape and a size, etc. of elements in a drawing may be exaggerated for a clearer description. A detailed description on exemplary embodiments described below refers to an accompanying drawing which shows a specific embodiment as an example. These embodiments are described in detail so that those skilled in the pertinent art can implement an embodiment. It should be understood that a variety of embodiments are different each other, but they do not need to be mutually exclusive. For example, a specific shape, structure and characteristic described herein may be implemented in other embodiment without departing from a scope and a spirit of the present disclosure in connection with an embodiment. In addition, it should be understood that a position or an arrangement of an individual element in each disclosed embodiment may be changed without departing from a scope and a spirit of an embodiment. Accordingly, a detailed description described below is not taken as a limited meaning and a scope of exemplary embodiments, if properly described, are limited only by an accompanying claim along with any scope equivalent to that claimed by those claims.

In the present disclosure, a term such as first, second, etc. may be used to describe a variety of elements, but the elements should not be limited by the terms. The terms are used only to distinguish one element from other element. For example, without getting out of a scope of a right of the present disclosure, a first element may be referred to as a second element and likewise, a second element may be also referred to as a first element. A term of and/or includes a combination of a plurality of relevant described items or any item of a plurality of relevant described items.

When an element in the present disclosure is referred to as being “connected” or “linked” to another element, it should be understood that it may be directly connected or linked to that another element, but there may be another element between them. Meanwhile, when an element is referred to as being “directly connected” or “directly linked” to another element, it should be understood that there is no another element between them.

As construction units shown in an embodiment of the present disclosure are independently shown to represent different characteristic functions, it does not mean that each construction unit is composed in a construction unit of separate hardware or one software. In other words, as each construction unit is included by being enumerated as each construction unit for convenience of a description, at least two construction units of each construction unit may be combined to form one construction unit or one construction unit may be divided into a plurality of construction units to perform a function, and an integrated embodiment and a separate embodiment of each construction unit are also included in a scope of a right of the present disclosure unless they are beyond the essence of the present disclosure.

A term used in the present disclosure is just used to describe a specific embodiment, and is not intended to limit the present disclosure. A singular expression, unless the context clearly indicates otherwise, includes a plural expression. In the present disclosure, it should be understood that a term such as “include” or “have”, etc. is just intended to designate the presence of a feature, a number, a step, an operation, an element, a part or a combination thereof described in the present specification, and it does not exclude in advance a possibility of presence or addition of one or more other features, numbers, steps, operations, elements, parts or their combinations. In other words, a description of “including” a specific configuration in the present disclosure does not exclude a configuration other than a corresponding configuration, and it means that an additional configuration may be included in a scope of a technical idea of the present disclosure or an embodiment of the present disclosure.

Some elements of the present disclosure are not a necessary element which performs an essential function in the present disclosure and may be an optional element for just improving performance. The present disclosure may be implemented by including only a construction unit which is necessary to implement essence of the present disclosure except for an element used just for performance improvement, and a structure including only a necessary element except for an optional element used just for performance improvement is also included in a scope of a right of the present disclosure.

Hereinafter, an embodiment of the present disclosure is described in detail by referring to a drawing. In describing an embodiment of the present specification, when it is determined that a detailed description on a relevant disclosed configuration or function may obscure a gist of the present specification, such a detailed description is omitted, and the same reference numeral is used for the same element in a drawing and an overlapping description on the same element is omitted.

In the present disclosure, a method and apparatus for personalizing walking ability data by measuring and analyzing steps using cameras and/or sensors will be proposed.

Additionally, the proposed method in the present disclosure may include determining abnormal signs of health according to the user's gait acquired over a certain period of time, or analyzing the user's footsteps to improve walking ability and providing guide footprints for improved step training using projection equipment.

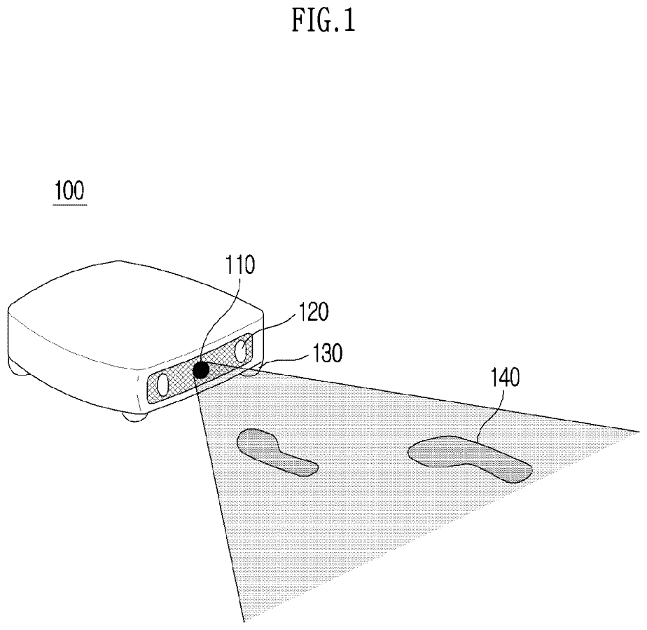

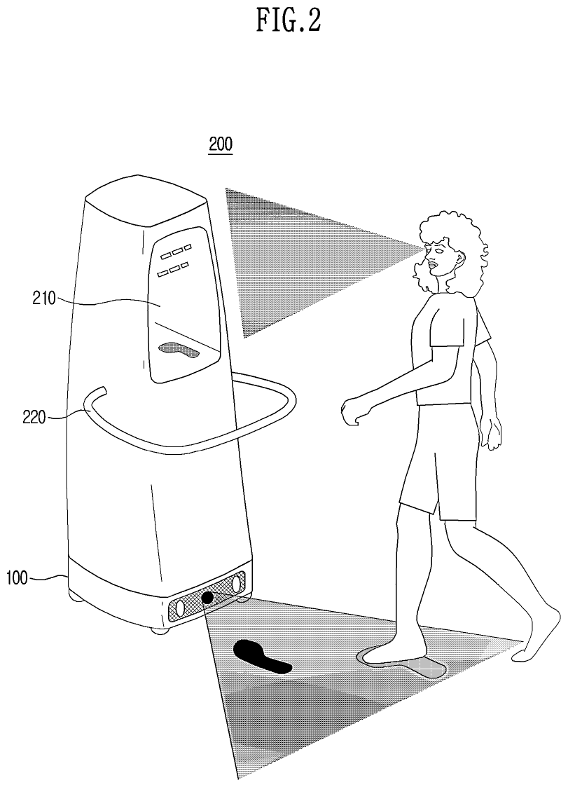

In this regard, the guide robot system for gait posture correction and evaluation proposed in the present disclosure may be provided in the form of FIG. 1 or FIG. 2.

FIG. 1 illustrates an example of a guide robot system for gait posture correction and evaluation according to an embodiment of the present disclosure.

Referring to FIG. 1, the gait guide robot 100 may include a projection device 110, a camera device 120, and a device 130 for movement.

For example, movement of the gait guide robot 100 may be performed using a device 130 for movement (e.g., wheels, etc.), and may be controlled omnidirectionally according to the user's gait.

The projection device 110 mounted on the gait guide robot 100 may correspond to equipment capable of projecting guide footprints 140.

The camera device mounted on the gait guide robot 100 may be used to evaluate whether the user appropriately stepped on the guide footsteps. For example, the camera device may correspond to a red/green/blue (RGB) camera, an infrared ray (IR) camera, a depth/distance camera, a stereoscopic camera, etc.

The form of the gait guide robot illustrated in FIG. 1 may exemplify a form in which the interface is minimized.

In the case of the gait guide robot illustrated in FIG. 1, information input, operation, and analysis results may be obtained using a terminal (eg, a smartphone, etc.) for setting personalized body information.

FIG. 2 illustrates another example of a guide robot system for gait posture correction and evaluation according to an embodiment of the present disclosure.

Referring to FIG. 2, the gait guide robot 200 may have a display 210 and a handle 220 added to the gait guide robot 100 illustrated in FIG. 1.

The display 210 may correspond to a touch-enabled display and may be a device for providing an interface to a user. Here, the interface may be intended to provide input of personalized body information and operation and analysis of the system.

The handle 220 may be a device to assist the user's walking motion.

In the case of the gait guide robot form illustrated in FIG. 2, Input of personalized body information and operation and analysis of the system may be performed using the terminal or the touch display provided by the main body.

Additionally, the form of the gait guide robot illustrated in FIG. 2 may be intended to guide the user's posture to a more correct posture. For example, through a display placed in front of the user, the user may look straight ahead without looking at the guide sole projected on the floor and receive feedback on the status of the user's walking.

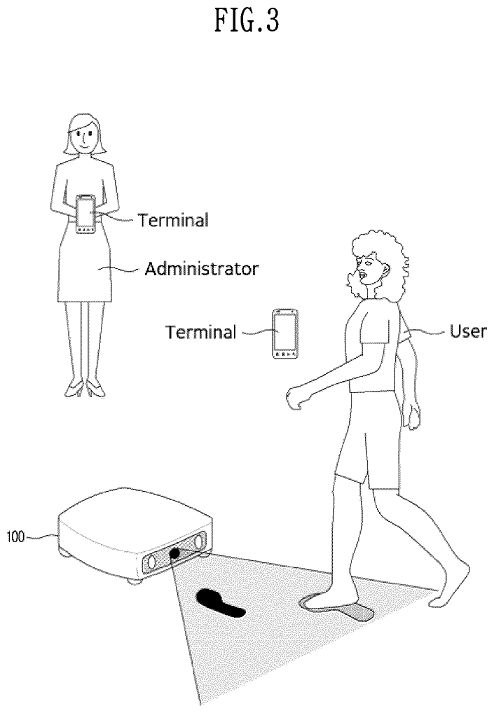

FIG. 3 illustrates a control method of a guide robot system according to an embodiment of the present disclosure.

Referring to FIG. 3, in order to control the gait guide robot system, a method in which the user directly uses the system using a terminal may be considered.

Alternatively, considering users who have difficulty using the terminal directly, a method in which a professional administrator sets up and operates the system for one or more users and supports the use of the system by that user group may be considered.

In FIG. 3, the form of the gait guide robot 100 illustrated in FIG. 1 is explained as an example. Substitute application in the form of the gait guide robot 200 illustrated in FIG. 2 is not excluded.

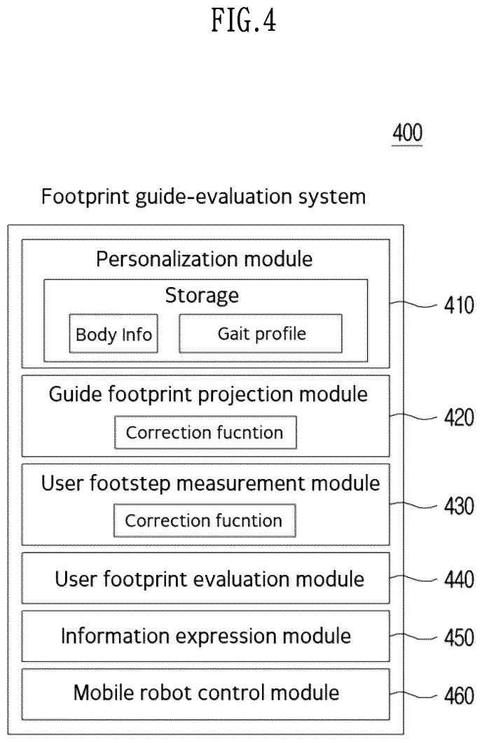

FIG. 4 illustrates a block diagram of a footstep guide-evaluation system by a guide robot according to an embodiment of the present disclosure.

Referring to FIG. 4, the footstep guide-evaluation system 400 proposed in the present disclosure may include a personalization module 410, a guide footprint projection module 420, a user footstep measurement module 430, a user footstep evaluation module 440, an information expression module 450, and a mobile robot control module 460.

The six modules illustrated in FIG. 4 are examples for clarity of explanation, for providing functions according to the proposed method of this disclosure, it is not excluded that some modules are merged with other modules to form one module, or that some modules are omitted.

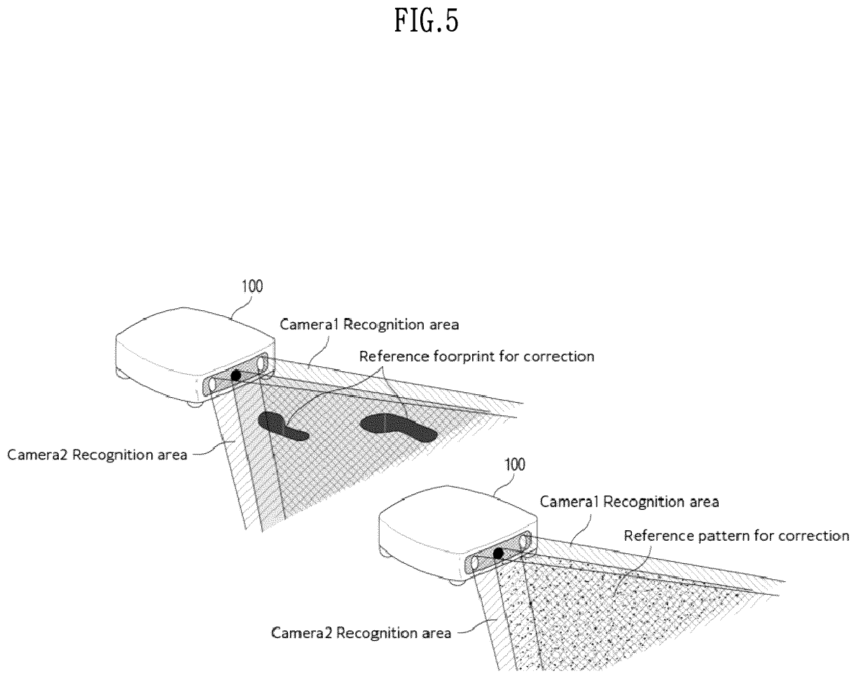

Hereinafter, the function of each module illustrated in FIG. 4 will be described in detail through examples of FIGS. 5 to 9.

The personalization module 410 may perform a function of processing personalized data such as the user's body information and gait profile. In this regard, the personalization module 410 may control storage and/or a database (DB) for managing personalized data.

The guide footprint projection module 420 may perform the function of projecting guide footprints to be provided to the user for gait correction.

The user footstep measurement module 430 may perform a function of measuring information on the footsteps of a user walking within the recognition area of the camera mounted on the gait guide robot.

In this regard, the guide footprint projection module 420 and the user footsteps measurement module 430 may have correction functions related to providing guide footsteps and measuring user footsteps.

FIG. 5 illustrates a correction function for providing guide footprints and measuring user footsteps according to an embodiment of the present disclosure.

Referring to FIG. 5, the correction function may use a plurality of camera devices and projection devices mounted on the gait guide robot 100.

For example, the first camera and the second camera may recognize the guide footprint projected/provided by the projection device, that is, the reference footprint for correction, as an image. In this regard, a reference pattern for correction as illustrated in FIG. 5 may be formed.

Based on this, the gait guide robot 100 may independently perform a correction function using the reference footprint for correction and/or the reference pattern for correction.

In FIG. 5, the form of the gait guide robot 100 illustrated in FIG. 1 is explained as an example, but substitute application in the form of the gait guide robot 200 illustrated in FIG. 2 is not excluded.

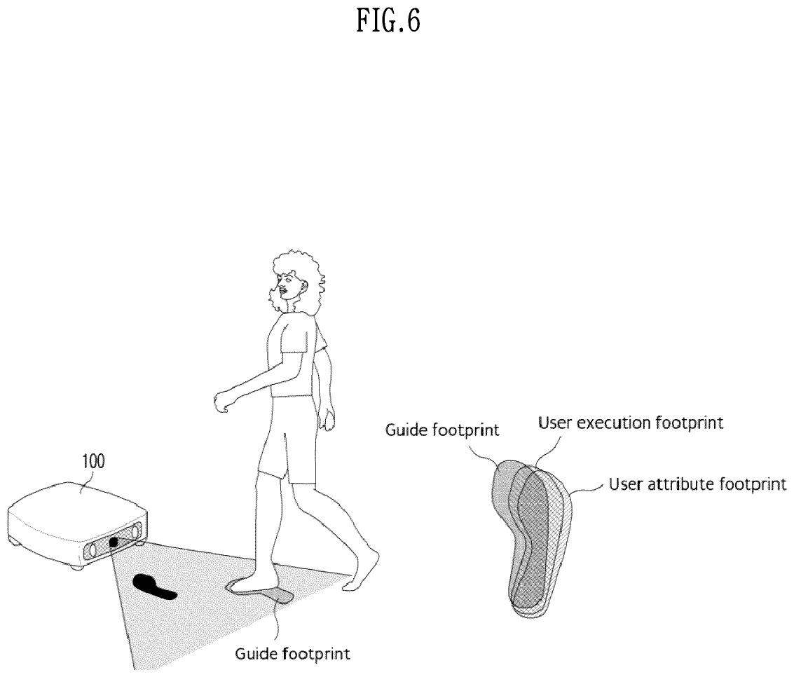

FIG. 6 illustrates a method of measuring guide footprints and user footprints for gait posture correction and evaluation according to an embodiment of the present disclosure.

The guide footprints described in FIG. 6 may be footprints projected by the guide footprint projection module 420 of FIG. 4, and user footprint measurement may be performed by the user footstep measurement module 430 of FIG. 4.

Referring to FIG. 6, in order to correctly correct the user's gait, the gait guide robot 100 may determine the hit area and direction of the user's footprints (i.e., user execution footprint) based on the projected guide footprints.

Here, the hit area may mean an overlapping area between the guide footprint and the user execution footprint.

The gait guide robot 100 may be designed to have a function that may perform scoring for each user's gait based on the determined hit area and direction. In other words, the gait guide robot 100 may measure the user's footprints and calculate a score for each gait by comparing the measured footprints with the guide footprints.

To evaluate gait, information on user attribute footprints and guide footprints corrected by analyzing the user's information for correct walking may be used.

Here, the user attribute footprint may correspond to a footprint profiled in order to train the user's gait to be a correct gait. As an example, the user attribute footprint may correspond to the user's footstep characteristics recorded when building a gait profile in the personalization module 410 of FIG. 4.

Guide footprints corrected as described above are drawn on the floor by the guide footprint estimation module 420 of FIG. 4, and the user tries to step on the guide footprints. At this time, the user footstep measurement module 430 of the gait guide robot 100 may be controlled to recognize the position of the foot performed by the user as an image through a camera, etc., and the recognized image may be used for gait evaluation.

When the user takes one step, the user evaluation module 440 of the gait guide robot 100 analyzes whether gait is improved using the performed foot position, and records the analyzed results/information. The gait guide robot 100 may have the function of analyzing trends during the overall training time by repeatedly performing such movements over a certain period of time.

In FIG. 6, the form of the gait guide robot 100 illustrated in FIG. 1 is explained as an example, but substitute application in the form of the gait guide robot 200 illustrated in FIG. 2 is not excluded.

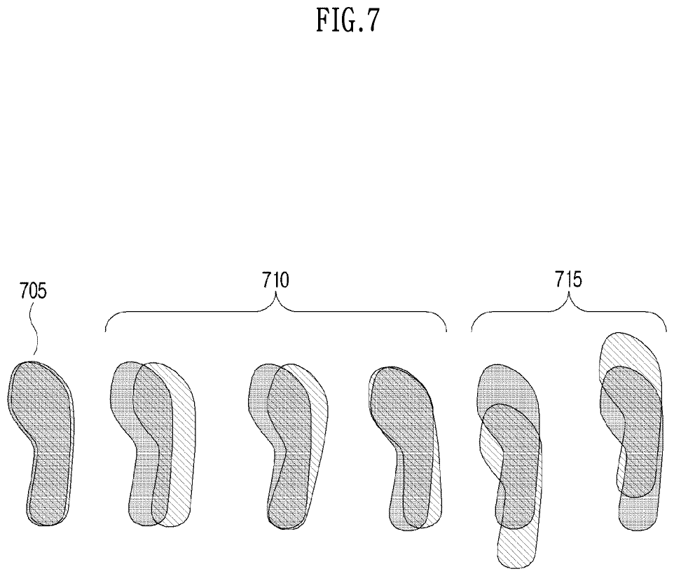

FIG. 7 illustrates an overlap state between guide footprints and user footprints according to an embodiment of the present disclosure.

Referring to FIG. 7, the gait guide robot 100 may measure/determine the state in which footsteps performed by the user overlap with the provided guide footprints.

The first footprint 705 illustrates a case where the user's execution footprint is correctly matched to the guide footprint.

Additionally, the second to fourth footprints 710 illustrate a case where a gap/offset occurs in the user's execution footprints to the left or right compared to the guide footprints.

Additionally, the fifth and sixth footprints 715 illustrate a case where a gap/offset occurs in the user's execution footprints forward or backward compared to the guide footprints.

In addition to the example illustrated in FIG. 7, various types of cases may occur regarding the overlap of guide footprints and user's footprints.

In this regard, the front or rear gap/offset may be caused by an error in the user's stride length or walking speed. In this case, a method of helping the user's gait training by correcting the movement of the guide robot may be considered. Additionally, the movement and speed of the calibrated guide robot may be used to analyze the user's walking characteristics.

The movements of the above-described guide robot may be controlled/performed by the mobile robot control module 460 of FIG. 4.

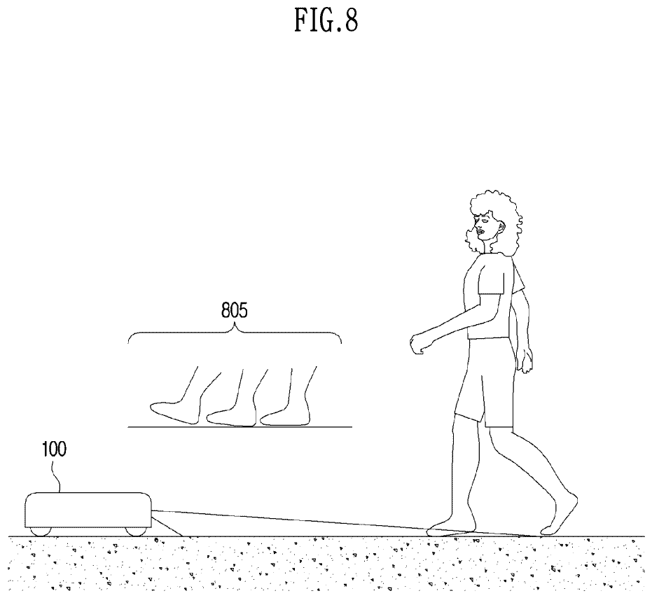

FIG. 8 illustrates footstep analysis according to an embodiment of the present disclosure.

For analyzing the user's walking state, the foot entry form 805 illustrated in FIG. 8 is a diagram of the foot entry form through a camera to analyze problems with foot drag or stepping.

For example, based on the foot entry form 805 confirmed through the camera, the entry angle of the foot, the time of occupying the ground, and/or the drag of the foot when the user walks may be analyzed through a video/image, etc.

In relation to the above-described method, the gait guide robot 100 may provide graphic effects to provide emotional feedback to the user according to the hit rate of the guide footprint. For example, the graphic effect may correspond to animation or color.

Provision of graphic effects may be controlled/performed by the information expression module 450 of FIG. 4.

For example, a pre-configured footprint animation effect (e.g., color/smear effect, etc.) may be provided to the user's profile.

Additionally or alternatively, based on the contents of the indoor interlocking system (e.g., forest path, flower path, season, weather, etc.), special footprints effects may be provided through images and sounds depending on the level of fidelity with which the user follows the guide footprints.

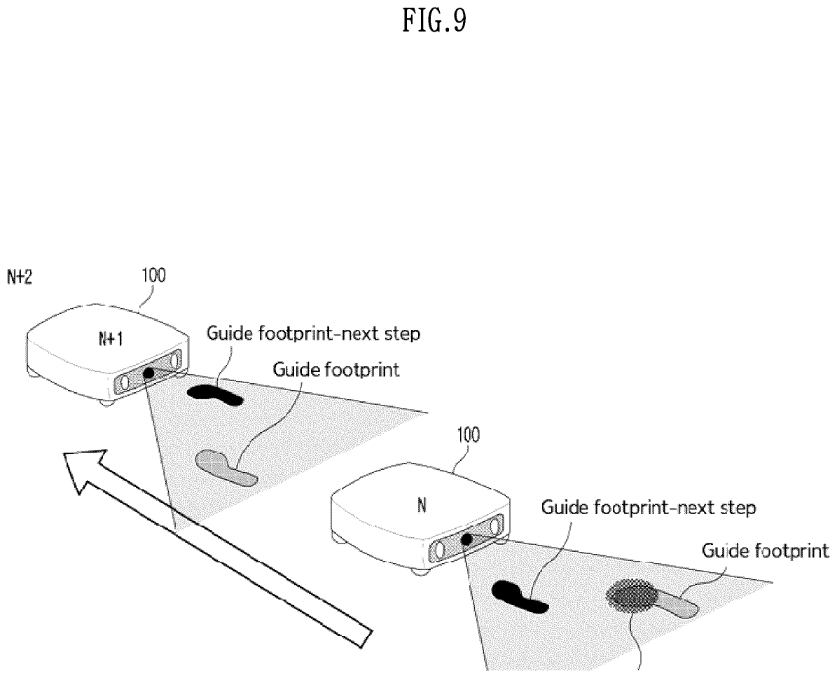

FIG. 9 illustrates guide footprint projection and movement of the gait guide robot according to an embodiment of the present disclosure.

Referring to FIG. 9, the gait guide robot 100 may move and provide/project guide footprints, and then, the user may walk to correct the gait posture. As an example, the gait guide robot 100 may sequentially move to the N position, N+1 position, and N+2 position and project guide footprints.

Specifically, the gait guide robot 100 completes guidance and evaluation of one step, and may express guide footprint-graphic effects based on this. Afterwards, for the next step, the gait guide robot 100 moves according to the user's direction and may express information for the next step (e.g., guide footsteps-next step).

Additionally or alternatively, the gait guide robot proposed in the present disclosure may be designed to have a cleaning function as well as the gait posture correction and evaluation functions described above.

In this case, gait posture correction and evaluation may be performed and cleaning of the area in which the gait guide robot moves may be performed at the same time.

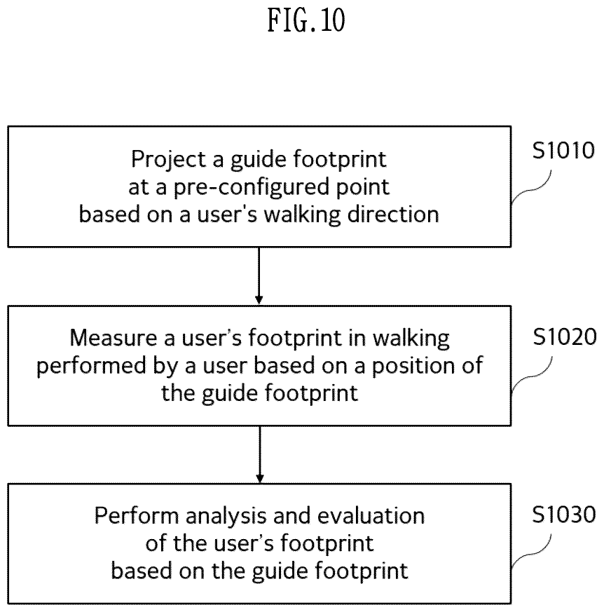

FIG. 10 illustrates an operational flowchart of a method for analyzing and evaluating a user's gait according to an embodiment of the present disclosure.

The operation flowchart of FIG. 10 may be based on the operation of the gait guide robot described through FIGS. 1 to 9 in the present disclosure.

In step S1010, the gait guide robot may project guide footprints at a pre-configured point based on the user's walking direction.

Here, the projected guide footprints may be determined based on gait data pre-stored/recorded for the user.

For example, the pre-stored/recorded gait data may correspond to data profiled based on one or more of data on the user's body or data on gait correction previously performed by the user. In this regard, the data on the user's body may be input through the interface of a display mounted on a device that projects the guide footprints (i.e., a gait guide robot) or through the interface of a terminal linked to the device (e.g., referrring to FIGS. 2 and 3).

Additionally or alternatively, the function of projecting guide footprints may be designed to be correctable based on pre-configured reference footprints and image recognition by one or more cameras (e.g., referring to FIG. 5).

In step S1020, the gait guide robot may measure the user's footprints in walking performed by the user based on the position of the corresponding guide footprints.

In this regard, the function of measuring guide footprints may be designed to be correctable based on a preset reference pattern and image recognition by one or more cameras (e.g., referring to FIG. 5).

In step S1030, the gait guide robot may perform analysis and evaluation of the user's footprints based on the guide footprints.

Here, the analysis and evaluation may be based on a scoring method performed on a step-by-step basis according to the overlap/hit area and direction of the user's footprints based on the guide footprints (e.g., referring to FIG. 7). That is, the scoring method may be performed for every gait/step.

In this regard, analysis of the user's footprint may be performed by additionally applying one or more of the entry angle of the user's foot, time of occupying the ground, or drag information (i.e., information on the drag of the foot on the ground) (e.g., referring to FIG. 8).

In this regard, the gait guide robot may move to another point in front of the user through a pre-configured movement control method according to the user's walking direction. By moving in front of the user, the direction of the footprints may be measured/analyzed more clearly, and the movement direction of the gait guide robot may be flexibly determined/changed by considering the user's movement direction.

Thereafter, the gait guide robot may project subsequent guide footprints at that point and measure the subsequent user footprints, thereby performing analysis and evaluation of the subsequent user footprints.

Additionally or alternatively, in connection with the operation of the gait guide robot, pre-configured special effects (e.g., graphic effects, video/audio effects, etc.) may be additionally provided for the corresponding guide footprints (e.g., referring to FIG. 9), depending on the degree of match between the guide footprints and the user's footprints. In this regard, pre-configured special effects may be configured in connection with gait data pre-stored for the user or in connection with content of an indoor linkage system.

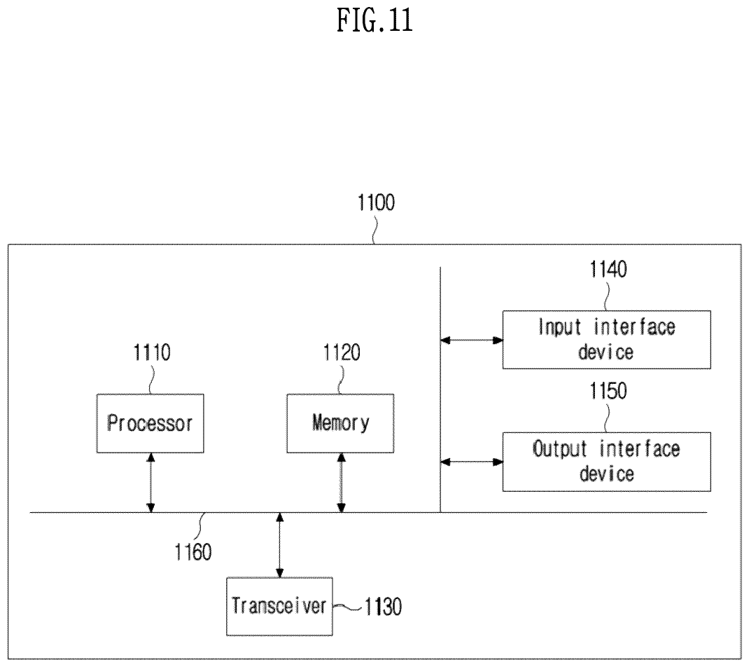

FIG. 11 is a block diagram illustrating an apparatus according to an embodiment of the present disclosure.

Referring to FIG. 11, a device 1100 may represent a device in which a method for performing analysis and evaluation for a user's gait described in the present disclosure is implemented.

For example, the device 1100 may generally support/perform function to profile information on the user's gait, function to project guide footprints, function to measure user's footprints, function to evaluate user's footprints, function to express information/effects for footprints, function to control the movement of the gait guide robot, etc.

The device 1100 may include at least one of a processor 1110, a memory 1120, a transceiver 1130, an input interface device 1140, and an output interface device 1150. Each of the components may be connected by a common bus 1160 to communicate with each other. In addition, each of the components may be connected through a separate interface or a separate bus centering on the processor 1110 instead of the common bus 1160.

The processor 1110 may be implemented in various types such as an application processor (AP), a central processing unit (CPU), a graphic processing unit (GPU), etc., and may be any semiconductor device that executes a command stored in the memory 1120. The processor 1110 may execute a program command stored in the memory 1120. The processor 1110 may be configured to implement a method for generating MPI view plane data and a method for filling view value blank areas based on FIGS. 1 to 10 described above.

And/or, the processor 1110 may store a program command for implementing at least one function for the corresponding modules in the memory 1120 and may control the operation described based on FIGS. 1 to 10 to be performed.

For example, the processor 1110 may control operations based on one or more modules described in FIG. 4 to be performed.

The memory 1120 may include various types of volatile or non-volatile storage media. For example, the memory 1120 may include read-only memory (ROM) and random access memory (RAM). In an embodiment of the present disclosure, the memory 1120 may be located inside or outside the processor 1110, and the memory 1120 may be connected to the processor 1110 through various known means.

The transceiver 1130 may perform a function of transmitting and receiving data processed/to be processed by the processor 1110 with an external device and/or an external system.

The input interface device 1140 is configured to provide data to the processor 1110.

The output interface device 1150 is configured to output data from the processor 1110.

According to the present disclosure, a method and appratus for controlling and configuring a guide robot system for correction and evaluation of walking posture, particularly gait, may be provided.

According to the present disclosure, a method and apparatus may be provided that can profile a user's gait and immediately train an improved gait to improve or maintain walking ability.

According to the present disclosure, it has the effect of achieving the purpose of maintaining body balance by maintaining correct walking posture and supporting the recognition of diseases or physical ability decline by analyzing accumulated walking postures and providing evaluation results of suspected diseases or physical abilities.

According to the present disclosure, it is possible to correct and evaluate the gait of each user while wearing the shoes they are wearing, by providing a structure that does not use the shape of a shoe or a pressure plate.

The components described in the example embodiments may be implemented by hardware components including, for example, at least one digital signal processor (DSP), a processor, a controller, an application-specific integrated circuit (ASIC), a programmable logic element, such as an FPGA, GPU other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The method according to example embodiments may be embodied as a program that is executable by a computer, and may be implemented as various recording media such as a magnetic storage medium, an optical reading medium, and a digital storage medium.

Various techniques described herein may be implemented as digital electronic circuitry, or as computer hardware, firmware, software, or combinations thereof. The techniques may be implemented as a computer program product, i.e., a computer program tangibly embodied in an information carrier, e.g., in a machine-readable storage device (for example, a computer-readable medium) or in a propagated signal for processing by, or to control an operation of a data processing apparatus, e.g., a programmable processor, a computer, or multiple computers.

A computer program(s) may be written in any form of a programming language, including compiled or interpreted languages and may be deployed in any form including a stand-alone program or a module, a component, a subroutine, or other units suitable for use in a computing environment. A computer program may be deployed to be executed on one computer or on multiple computers at one site or distributed across multiple sites and interconnected by a communication network.

Processors suitable for execution of a computer program include, by way of example, both general and special purpose microprocessors, and any one or more processors of any kind of digital computer. Generally, a processor will receive instructions and data from a read-only memory or a random access memory or both. Elements of a computer may include at least one processor to execute instructions and one or more memory devices to store instructions and data. Generally, a computer will also include or be coupled to receive data from, transfer data to, or perform both on one or more mass storage devices to store data, e.g., magnetic, magneto-optical disks, or optical disks. Examples of information carriers suitable for embodying computer program instructions and data include semiconductor memory devices, for example, magnetic media such as a hard disk, a floppy disk, and a magnetic tape, optical media such as a compact disk read only memory (CD-ROM), a digital video disk (DVD), etc. and magneto-optical media such as a floptical disk, and a read only memory (ROM), a random access memory (RAM), a flash memory, an erasable programmable ROM (EPROM), and an electrically erasable programmable ROM (EEPROM) and any other known computer readable medium. A processor and a memory may be supplemented by, or integrated into, a special purpose logic circuit.

The processor may run an operating system (OS) and one or more software applications that run on the OS. The processor device also may access, store, manipulate, process, and create data in response to execution of the software. For purpose of simplicity, the description of a processor device is used as singular; however, one skilled in the art will be appreciated that a processor device may include multiple processing elements and/or multiple types of processing elements. For example, a processor device may include multiple processors or a processor and a controller. In addition, different processing configurations are possible, such as parallel processors.

Also, non-transitory computer-readable media may be any available media that may be accessed by a computer, and may include both computer storage media and transmission media.

The present specification includes details of a number of specific implements, but it should be understood that the details do not limit any invention or what is claimable in the specification but rather describe features of the specific example embodiment. Features described in the specification in the context of individual example embodiments may be implemented as a combination in a single example embodiment. In contrast, various features described in the specification in the context of a single example embodiment may be implemented in multiple example embodiments individually or in an appropriate sub-combination. Furthermore, the features may operate in a specific combination and may be initially described as claimed in the combination, but one or more features may be excluded from the claimed combination in some cases, and the claimed combination may be changed into a sub-combination or a modification of a sub-combination.

Similarly, even though operations are described in a specific order on the drawings, it should not be understood as the operations needing to be performed in the specific order or in sequence to obtain desired results or as all the operations needing to be performed. In a specific case, multitasking and parallel processing may be advantageous. In addition, it should not be understood as requiring a separation of various apparatus components in the above described example embodiments in all example embodiments, and it should be understood that the above-described program components and apparatuses may be incorporated into a single software product or may be packaged in multiple software products.

It should be understood that the example embodiments disclosed herein are merely illustrative and are not intended to limit the scope of the invention. It will be apparent to one of ordinary skill in the art that various modifications of the example embodiments may be made without departing from the spirit and scope of the claims and their equivalents.

Accordingly, it is intended that this disclosure embrace all other substitutions, modifications and variations belong within the scope of the following claims.

What is claimed is:

1. A method of analyzing and evaluating gait, the method comprising:
projecting a guide footprint at a pre-configured point based on a user's walking direction;
measuring a user's footprint in walking performed by a user based on a position of the guide footprint; and
performing analysis and evaluation of the user's footprint based on the guide footprint,
wherein the guide footprint is determined based on gait data pre-stored for the user, and
wherein the analysis and evaluation of the user's footprint is based on a scoring method performed on a step-by-step basis according to the overlapping area of the user's footprint based on the guide footprint and a direction of the user's footprint.

2. The method of claim 1, further comprising:
moving to other point in front of the user using a pre-configured movement control method according to walking direction of the user; and
performing analysis and evaluation of a user's subsequent footprint by projecting a subsequent guide footprint and measuring the user's subsequent footprint at the other point.

3. The method of claim 1,
wherein the pre-stored gait data corresponds to data profiled based on one or more of data on a body of the user or data on gait correction previously performed by the user.

4. The method of claim 3,
wherein the data on a body of the user is input through an interface of a display mounted on a device that projects the guide footprint or an interface of a terminal linked to the device.

5. The method of claim 1,
wherein a function of projecting the guide footprint is designed to enable correction based on pre-configured reference footprints and image recognition by one or more cameras.

6. The method of claim 1,
wherein a function of measuring the guide footprint is designed to enable correction based on a pre-configured reference pattern and image recognition by one or more cameras.

7. The method of claim 1,
wherein the analysis of the user's footprint is performed by additionally applying one or more of an entry angle, time of occupying a ground, or drag information of foot of the user.

8. The method of claim 1,
wherein a pre-configured special effect is additionally provided for the guide footprint depending on the degree of coincidence between the guide footprint and the user's footprint.

9. The method of claim 8,
wherein the pre-configured special effect is configured in association with gait data pre-stored for the user or content of an indoor interlocking system.

10. An apparatus for analyzing and evaluating gait, the apparatus comprising:
a processor and a memory,
wherein the processor is configured to:
project a guide footprint at a pre-configured point based on a user's walking direction;
measure a user's footprint in walking performed by a user based on a position of the guide footprint; and
perform analysis and evaluation of the user's footprint based on the guide footprint,
wherein the guide footprint is determined based on gait data pre-stored for the user, and
wherein the analysis and evaluation of the user's footprint is based on a scoring method performed on a step-by-step basis according to the overlapping area of the user's footprint based on the guide footprint and a direction of the user's footprint.

11. The apparatus of claim 10, wherein the processor is configured to:
move to other point in front of the user using a pre-configured movement control method according to walking direction of the user; and
perform analysis and evaluation of a user's subsequent footprint by projecting a subsequent guide footprint and measuring the user's subsequent footprint at the other point.

12. The apparatus of claim 10,
wherein the pre-stored gait data corresponds to data profiled based on one or more of data on a body of the user or data on gait correction previously performed by the user.

13. The apparatus of claim 12,
wherein the data on a body of the user is input through an interface of a display mounted on a device that projects the guide footprint or an interface of a terminal linked to the device.

14. The apparatus of claim 10,
wherein a function of projecting the guide footprint is designed to enable correction based on pre-configured reference footprints and image recognition by one or more cameras.

15. The apparatus of claim 10,
wherein a function of measuring the guide footprint is designed to enable correction based on a pre-configured reference pattern and image recognition by one or more cameras.

16. The apparatus of claim 10,
wherein the analysis of the user's footprint is performed by additionally applying one or more of an entry angle, time of occupying a ground, or drag information of foot of the user.

17. The apparatus of claim 10,
wherein a pre-configured special effect is additionally provided for the guide footprint depending on the degree of coincidence between the guide footprint and the user's footprint.

18. The apparatus of claim 17,
wherein the pre-configured special effect is configured in association with gait data pre-stored for the user or content of an indoor interlocking system.

19. One or more non-transitory computer readable medium storing one or more instructions,
wherein the one or more instructions are executed by one or more processors and control an apparatus for analyzing and evaluating gait to:
project a guide footprint at a pre-configured point based on a user's walking direction;
measure a user's footprint in walking performed by a user based on a position of the guide footprint; and
perform analysis and evaluation of the user's footprint based on the guide footprint,
wherein the guide footprint is determined based on gait data pre-stored for the user, and
wherein the analysis and evaluation of the user's footprint is based on a scoring method performed on a step-by-step basis according to the overlapping area of the user's footprint based on the guide footprint and a direction of the user's footprint.

20. The computer readable medium of claim 19,
wherein the one or more instructions control to:
move to other point in front of the user using a pre-configured movement control method according to walking direction of the user; and
perform analysis and evaluation of a user's subsequent footprint by projecting a subsequent guide footprint and measuring the user's subsequent footprint at the other point.
